SEQUENCE LISTING (11 sequences)

SEQ ID NO: 1 - protein, 9 aa, Artificial Sequence
  source: /note="Description of Artificial Sequence: Synthetic peptide"
GFPRFAGFP

SEQ ID NO: 2 - protein, 21 aa, Artificial Sequence
  source: /note="Description of Artificial Sequence: Synthetic peptide"
FKEFAKLFKEFAKLFHKFAKL

SEQ ID NO: 3 - protein, 21 aa, Artificial Sequence
  source: /note="Description of Artificial Sequence: Synthetic peptide"
LKELAKLLKELAKLLHELAKL

SEQ ID NO: 4 - protein, 7 aa, Artificial Sequence
  source: /note="Description of Artificial Sequence: Synthetic peptide"
KGKGKGK

SEQ ID NO: 5 - protein, 12 aa, Artificial Sequence
  source: /note="Description of Artificial Sequence: Synthetic peptide"
LRALLRALLRAL

SEQ ID NO: 6 - protein, 14 aa, Artificial Sequence
  source: /note="Description of Artificial Sequence: Synthetic peptide"
KLKLKLKLKLKLKL

SEQ ID NO: 7 - protein, 24 aa, Artificial Sequence
  source: /note="Description of Artificial Sequence: Synthetic peptide"
LRLRLRLRLRLFPVRLRLRLRLRL

SEQ ID NO: 8 - DNA, 21 bp, Artificial Sequence
  source: /note="Description of Artificial Sequence: Synthetic oligonucleotide"
gctaaaaagagagagagatcg

SEQ ID NO: 9 - DNA, 21 bp, Artificial Sequence
  source: /note="Description of Artificial Sequence: Synthetic oligonucleotide"
cgatctctctctctttttagc

SEQ ID NO: 10 - protein, 29 aa, Artificial Sequence
  source: /note="Description of Artificial Sequence: Synthetic peptide"
ELKAIAQEFKAIAKEFKAIAFEFKAIAQK

SEQ ID NO: 11 - DNA, 25 bp, Artificial Sequence
  source: /note="Description of Artificial Sequence: Synthetic oligonucleotide"
gggaaaggaaacttaaaaaaaaggg

(19) United States
(12) Patent Application Publication
(10) Pub. No.: US 2023/0295711 A1
(43) Pub. Date: Sep. 21, 2023

(54) COMPOSITIONS AND METHODS FOR SEQUENCING USING AT LEAST ELECTRICAL CHARACTERISTICS

(71) Applicant: Illumina, Inc., San Diego, CA (US)

(72) Inventors: Jeffrey Mandell, San Diego, CA (US); Maria Candelaria Rogert Bacigalupo, Encinitas, CA (US)

(73) Assignee: Illumina, Inc., San Diego, CA (US)

(21) Appl. No.: 18/008,953

(22) PCT Filed: Jun. 24, 2021
(86) PCT No.: PCT/US2021/038887

§ 371 Date: Dec. 7, 2022

Related U.S. Application Data
(60) Provisional application No. 63/046,618, filed on Jun. 30, 2020.

(51) Int. Cl. C12Q 1/6869; G01N 27/327

(52) U.S. Cl. CPC C12Q 1/6869; G01N 27/3276

(57) ABSTRACT

Provided herein are compositions and methods for sequencing using at least altering electrical characteristics of polymer bridges. In some examples, the bridges may span the space between first and second electrodes and may include first and second polymer chains that are hybridized to one another. A plurality of nucleotides may be coupled to corresponding labels. A polymerase may be coupled to the bridge and may add nucleotides to a first polynucleotide using at least a sequence of a second polynucleotide. The labels corresponding to those nucleotides respectively may alter hybridization between the first and second polymer chains. Detection circuitry may detect a sequence in which the polymerase adds the nucleotides to the first polynucleotide using at least changes in an electrical signal through the bridge, the changes being responsive to the respective alterations of hybridization using the labels corresponding to those nucleotides.

### CROSS-REFERENCE TO RELATED APPLICATIONS

This application claims the benefit of U.S. Provisional Pat. Application No. 63/046,618, filed Jun. 30, 2020 and entitled “Compositions and Methods for Sequencing Using at Least Electrical Characteristics,” the entire contents of which are incorporated by reference herein.

### SEQUENCE LISTING

[0001.1] The instant application contains a Sequence Listing which has been submitted electronically in ASCII format and is hereby incorporated by reference in its entirety. Said ASCII copy, created on Jun. 11, 2021, is named IP_1969_PCT_SL.txt and is 3,590 bytes in size.

### BACKGROUND

A significant amount of academic and corporate time and energy has been invested into sequencing polynucleotides, such as DNA. Some sequencing systems use “sequencing by synthesis” (SBS) technology and fluorescence-based detection. However, fluorescence-based detection may require optical components such as excitation light sources, imaging devices, and the like, which may be complex, time-consuming to operate, and costly.

### SUMMARY

Examples provided herein are related to sequencing using at least altering electrical characteristics of bridges between electrodes. Compositions and methods for performing such sequencing are disclosed.

In some examples, the bridges may span the space between first and second electrodes and may include a single polymer chain, or may include first and second polymer chains that are hybridized to one another, or may include more than two polymer chains. A plurality of nucleotides may be coupled to corresponding labels. A polymerase may be coupled to, or in proximity to, the bridge and may add nucleotides to a first polynucleotide using at least a sequence of a second polynucleotide. The labels corresponding to those nucleotides respectively may alter an electrical characteristic of the bridge. Detection circuitry may detect a sequence in which the polymerase adds the nucleotides to the first polynucleotide using at least changes in an electrical signal, for example current or voltage, through the bridge, the changes being responsive to the respective alterations of the electrical characteristic using the labels corresponding to those nucleotides.

Provided in some examples herein is a composition that includes first and second electrodes separated from one another by a space, and a bridge spanning the space between the first and second electrodes. The bridge may include first and second polymer chains hybridized to one another. The composition also may include first and second polynucleotides, and a plurality of nucleotides, each nucleotide coupled to a corresponding label. The composition also may include a polymerase to add nucleotides of the plurality of nucleotides to the first polynucleotide using at least a sequence of the second polynucleotide. The labels corresponding to those nucleotides respectively may alter an electrical characteristic of at least one of the first and second polymer chains. The composition may include detection circuitry to detect a sequence in which the polymerase adds the nucleotides to the first polynucleotide using at least changes in an electrical signal through the bridge. The changes may be responsive to alteration of the electrical characteristic using the labels corresponding to those nucleotides.

In some examples, the first and second polymer chains respectively include first and second polynucleotides hybridized to one another. In some examples, the labels include respective oligonucleotides that alter the hybridization between the first and second polynucleotides. In some examples, the oligonucleotides alter the hybridization in different locations than one another. In some examples, the oligonucleotides alter the hybridization in regions of different length.

In some examples, the polynucleotides of the first and second polymer chains and the oligonucleotides of the labels include non-naturally occurring DNA. In some examples, the non-naturally occurring DNA includes enantiomeric DNA. In some examples, the oligonucleotides include modified nucleotides. In some examples, the modified nucleotides have modified backbones, modified sugars, or modified bases. In some examples, the oligonucleotides include nucleic acid analogs selected from the group consisting of PNA and LNA.

In some examples, the first and second polynucleotides include DNA, and the labels include proteins that interact with the DNA. In some examples, the labels include DNA intercalators. In some examples, the labels include minor groove binders. In some examples, the labels include peptide intercalators. In some examples, the labels include intertwining alpha helices.

In some examples, the first and second polymer chains respectively include first and second polypeptides hybridized to one another. In some examples, each of the labels includes a protein, peptide, or intercalator that alters the hybridization between the first and second polypeptides.

Provided in some examples herein is a method. The method may include adding, using a polymerase, nucleotides to a first polynucleotide using at least a sequence of a second polynucleotide. The method may include altering, using labels respectively coupled to the nucleotides, an electrical characteristic of at least one of a first polymer chain and a second polymer chain of a bridge spanning a space between first and second electrodes. The method may include detecting a sequence in which the polymerase adds the nucleotides to the first polynucleotide using at least changes in electrical signal through the bridge that are responsive to respective alterations of the electrical characteristic using the labels corresponding to those nucleotides.

In some examples, the first and second polymer chains respectively include first and second polynucleotides hybridized to one another. In some examples, the labels include respective oligonucleotides that alter the hybridization between the first and second polynucleotides. In some examples, the labels alter the hybridization in different locations than one another. In some examples, the labels alter the hybridization in regions of different length. In some examples, the polynucleotides of the first and second polymer chains and the oligonucleotides of the labels include non-naturally occurring DNA. In some examples, the non-naturally occurring DNA includes enantiomeric DNA. In some examples, the oligonucleotides include modified nucleotides. In some examples, the modified nucleotides have modified backbones, modified sugars, or modified bases. In some examples, the oligonucleotides include nucleic acid analogs selected from the group consisting of PNA and LNA.

In some examples, the first and second polynucleotides include DNA, and wherein the labels include proteins that interact with the DNA. In some examples, the labels include DNA intercalators. In some examples, the labels include minor groove binders. In some examples, the labels include peptide intercalators. In some examples, the labels include intertwining alpha helices.

In some examples, the first and second polymer chains respectively include first and second polypeptide chains hybridized to one another. In some examples, each of the labels includes a protein, peptide, or intercalator that alters the hybridization between the first and second polypeptides.

Provided in some examples herein is a composition that includes first and second electrodes separated from one another by a space, and a bridge spanning the space between the first and second electrodes. The bridge may include a polymer chain. The composition may include first and second polynucleotides, and a plurality of nucleotides, each nucleotide coupled to a corresponding label. The composition may include a polymerase to add nucleotides of the plurality of nucleotides to the first polynucleotide using at least a sequence of the second polynucleotide. The labels corresponding to those nucleotides respectively may alter an electrical characteristic of the polymer chain. The composition may include detection circuitry to detect a sequence in which the polymerase adds the nucleotides to the first polynucleotide using at least changes in an electrical signal through the bridge. The changes may be responsive to alteration of the electrical characteristic using the labels corresponding to those nucleotides.

In some examples, the polymer chain includes a polypeptide chain. In some examples, the labels include peptide intercalators. In some examples, the labels include intertwining alpha helices.

Provided in some examples herein is a method for sequencing that includes adding, using a polymerase, nucleotides to a first polynucleotide using at least a sequence of a second polynucleotide. The method may include altering, using labels respectively coupled to the nucleotides, an electrical characteristic of a polymer chain of a bridge spanning a space between first and second electrodes. The method may include detecting a sequence in which the polymerase adds the nucleotides to the first polynucleotide using at least changes in electrical signal through the bridge that are responsive to respective alterations of the electrical characteristic using the labels corresponding to those nucleotides.

In some examples, the polymer chain includes a polypeptide chain. In some examples, the labels include peptide intercalators. In some examples, the labels include intertwining alpha helices.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate an example composition for sequencing that includes a double-stranded polymer bridge and nucleotide labels that alter an electrical characteristic of at least one of the polymer strands of the bridge.

FIGS. 2A-2C schematically illustrate examples of nucleotides with labels that alter an electrical characteristic of at least one of the polymer strands of a double-stranded polymer bridges.

FIG. 3 schematically illustrates an example composition for sequencing that includes a double-stranded polynucleotide bridge and nucleotide labels that alter hybridization between the polynucleotides of the bridge. Figure discloses SEQ ID NO: 11.

FIG. 4 illustrates an example flow of operations in a method for sequencing using a double-stranded polymer bridge and nucleotide labels that alter an electrical characteristic of at least one of the polymer strands of the bridge.

FIGS. 5A-5B schematically illustrate an example composition for sequencing that includes a single-stranded polymer bridge and nucleotide labels that alter an electrical characteristic of the bridge.

FIG. 6 illustrates an example flow of operations in a method for sequencing using a single-stranded polymer bridge and nucleotide labels that alter an electrical characteristic of the bridge.

FIGS. 7A-7C illustrate example polymer bridges including more than two polymer chains.

### DETAILED DESCRIPTION

Examples provided herein are related to sequencing using at least altering electrical characteristics of polymer chains. Compositions and methods for performing such sequencing are disclosed.

More specifically, the present compositions and methods suitably may have the benefits of being used to sequence polynucleotides in a manner that is robust, reproducible, sensitive, accurate, works in real time, detects single molecules, and has high throughput. For example, the present compositions can include first and second electrodes and a bridge that spans the space between the electrodes. The bridge can include double-stranded polymers, e.g., can include first and second polymer chains that are hybridized to one another in such a manner as to allow electrical current to flow from one electrode to another through the bridge, can include more than two polymer chains, or can include a single polymer chain that allows electrical current to flow from one electrode to another through the bridge. Labels, which may be coupled to respective nucleotides, may alter one or more electrical characteristics of the bridge, for example the electrical conductivity or electrical impedance of the bridge, and using at least such alteration the respective nucleotide may be identified.

First, some terms used herein will be briefly explained. Then, some example compositions and example methods for electronically sequencing polynucleotides will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially”, “approximately”, and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “electrode” is intended to mean a solid structure that conducts electricity. Electrodes may include any suitable electrically conductive material, such as gold, palladium, or platinum, or combinations thereof.

As used herein, the term “bridge” is intended to mean a structure that extends between, and couples to, two other structures. A bridge may span a space between other structures, such as between two electrodes. Not all elements of a bridge need to be directly coupled to both structures. For example, in a bridge that includes first and second polymer chains associated with one another and spanning the space between two electrodes, at least one end of one of the polymer chains is coupled to one of the electrodes, and at least one end of one of the polymer chains is coupled to the other electrode. However, both polymer chains need not be coupled to both of the electrodes, and indeed one of the polymer chains need not be coupled to either of the electrodes. A bridge may include multiple components which are coupled to one another in such a manner as to extend between, and collectively connect to, other structures. A bridge may be coupled to another structure, such as an electrode, via a chemical bond, e.g., via a covalent bond, hydrogen bond, ionic bond, dipole-dipole bond, London dispersion forces, or any suitable combination thereof.

As used herein, a “polymer” refers to a molecule including a chain of many subunits, that may be referred to as monomers, that are coupled to one another. The subunits may repeat, or may differ from one another. Polymers and their subunits can be biological or synthetic. Example biological polymers that suitably can be included within a bridge or a label include polynucleotides (made from nucleotide subunits), polypeptides (made from amino acid subunits), polysaccharides, polynucleotide analogs, and polypeptide analogs. Example polynucleotides and polynucleotide analogs suitable for use in a bridge or a label include DNA, enantiomeric DNA, RNA, PNA (peptide-nucleic acid), morpholinos, and LNA (locked nucleic acid). Polymers may include spacer subunits, derived from phosphoramidites, which may be coupled to polynucleotides but which lack nucleobases, such as commercially available from Glen Research (Sterling, VA), for example spacer phosphoramidite 18 (18-O-Dimethoxytritylhexaethyleneglycol,l-[(2-cyanoethyl)-(N,N-diisopropyl)]-phosphoramidite). Example synthetic polypeptides can include all natural amino acids, such as charged amino acids, hydrophilic, hydrophobic, and neutral amino acid residues. Example synthetic polymers that suitably can be included within a bridge or label include PEG (polyethylene glycol), PPG (polypropylene glycol), PVA (polyvinyl alcohol), PE (polyethylene), LDPE (low density polyethylene), HDPE (high density polyethylene), polypropylene, PVC (polyvinyl chloride), PS (polystyrene), NYLON (aliphatic polyamides), TEFLON® (tetrafluoroethylene), thermoplastic polyurethanes, polyaldehydes, polyolefins, poly(ethylene oxides), poly(co-alkenoic acid esters), poly(alkyl methacrylates), and other polymeric chemical and biological linkers such as described in Hermanson, Bioconjugate Techniques, third edition, Academic Press, London (2013).

As used herein, “hybridize” is intended to mean noncovalently associating a first polymer to a second polymer along the lengths of those polymers. For instance, two DNA polynucleotide strands may associate through complementary base pairing. The strength of the association between the first and second polymers increases with the complementarity between the sequences of monomer units within those polymers. For example, the strength of the association between a first polynucleotide and a second polynucleotide increases with the complementarity between the sequences of nucleotides within those polynucleotides.

As used herein, the term “nucleotide” is intended to mean a molecule that includes a sugar and at least one phosphate group, and in some examples also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide can be a single stranded sequence of nucleotides, such as RNA or single stranded DNA, a double stranded sequence of nucleotides, such as double stranded DNA, or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are example examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded polynucleotide template, and can sequentially add nucleotides to the growing primer to form a polynucleotide having a sequence that is complementary to that of the template.

As used herein, the term “primer” is defined as a polynucleotide to which nucleotides are added via a free 3′ OH group. A primer may have a 3′ block preventing polymerization until the block is removed. A primer can also have a modification at the 5′ terminus to allow a coupling reaction or to couple the primer to another moiety. The primer length can be any number of bases long and can include a variety of non-natural nucleotides.

As used herein, the term “label” is intended to mean a structure that couples to a bridge in such a manner as to cause a change in an electrical characteristic of the bridge, such as electrical impedance or electrical conductivity, and based upon which change the nucleotide may be identified. For example, a label may hybridize to a polymer chain within such a bridge, and the hybridization may cause an electrical conductivity or electrical impedance change of the bridge. Or, for example, a label may intercalate between polymer chains within such a bridge, and the intercalation may cause the electrical conductivity or electrical impedance change of the bridge. However, it should be appreciated that a label may alter any suitable electrical characteristic of a polymer chain within a bridge. In examples provided herein, labels can be coupled to nucleotides.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates can include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates can include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can comprise a single material or a plurality of different materials. Substrates can be composites or laminates. In some examples, the substrate comprises an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may comprise posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

### Example Compositions and Methods for Sequencing Polynucleotides

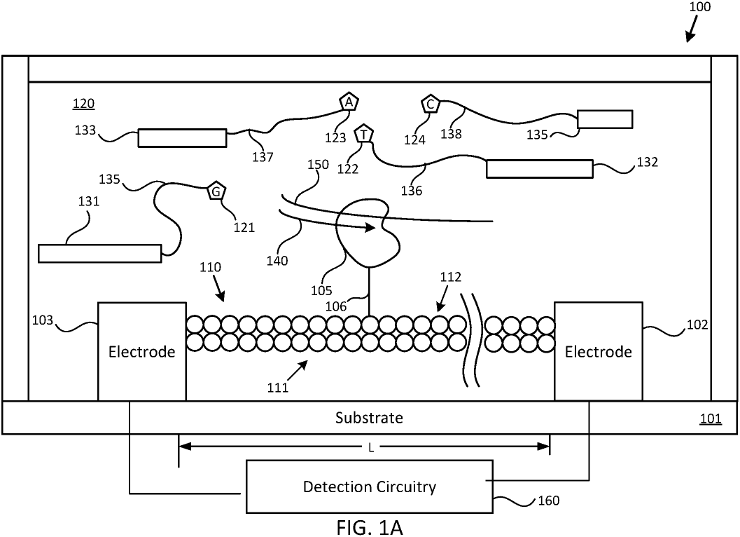

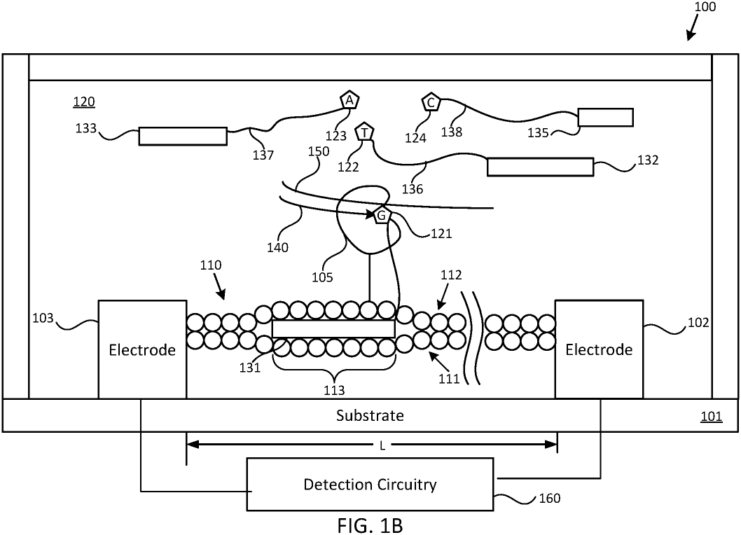

FIGS. 1A-1B illustrate an example composition 100 for sequencing that includes a double-stranded polymer bridge. Referring now to FIG. 1A, composition 100 includes substrate 101, first electrode 102, second electrode 103, polymerase 104, bridge 110, nucleotides 121, 122, 123, and 124, labels 131, 132, 133, and 134 respectively coupled to those nucleotides, first polynucleotide 140, second polynucleotide 150, and detection circuitry 160. Polymerase 105 is in proximity of bridge 110, and in some examples may be coupled to bridge 110 via linker 106 in a manner such as known in the art. Such linker chemistries include maleimide chemistry to reactive thiols on cysteine residues, NHS ester chemistry to reactive amines on lysine residues, biotin-Streptavidin, and Spytag-SpyCatcher, for example. In the example illustrated in FIGS. 1A-1B, components of composition 100 may be enclosed within a flow cell (e.g., having walls 161, 162, 162) filled with fluid 120 in which nucleotides 121, 122, 123, and 124 (with associated labels), polynucleotides 140, 150, and suitable reagents may be carried.

Substrate 101 may support first electrode 102 and second electrode 103. First electrode 102 and second electrode 103 may be separated from one another by a space, e.g., a space of length L as indicated in FIG. 1A. The value of L may be, in some examples, from about 1 nm to about 1 µm, e.g., from about 1 nm to about 100 nm, e.g., from about 1 nm to about 10 nm, e.g., from about 10 nm to about 25 nm, e.g., from about 25 nm to about 50 nm. First electrode 102 and second electrode 103 may have any suitable shape and arrangement, and are not limited to the approximately rectangular shape suggested in FIG. 1A. The sidewalls of first electrode 102 and second electrode 103 illustrated in FIG. 1A may be, but need not necessarily be, vertical or parallel to one another, and need not necessarily meet the top surfaces of such electrodes at a right angle. For example, first electrode 102 and second electrode 103 may be irregularly shaped, may be curved, or include any suitable number of obtuse or acute angles. In some examples, first electrode 102 and second electrode 103 may be arranged vertically relative to one another. The value L may refer to the spacing between the closest points of first electrode 102 and second electrode 103 to one another.

Bridge 110 may span the space between first electrode 102 and second electrode 103, and may include first polymer chain 111 and second polymer chain 112 hybridized to one another (the circles within the respective polymer chains being intended to suggest monomer units that are coupled to one another along the lengths of the polymer chains). First polymer chain 111 and second polymer chain 112 may include the same type of polymer, although the sequence of monomer units in the respective polymer chains may not necessarily be the same as one another. For example, first polymer chain 111 may have a sequence that is complementary to the sequence of second polymer chain 112. First and second polymer chains 111, 112, each may have length that is approximately the same as length L of the space between first electrode 102 and second electrode 103 or otherwise permits polymer chains 111, 112 to span the space between first electrode 102 and second electrode 103, e.g., such that first polymer chain 111 and second polymer chain 112 each may be coupled directly to each of first electrode 102 and second electrode 103 (e.g., via respective bonds). It should be understood that in some configurations, neither first polymer chain 111 nor second polymer chain 112 necessarily is coupled directly to one or both of first electrode 102 and second electrode 103. Instead, either or both of first polymer chain 111 and second polymer chain 112 may be directly coupled to one or more other structures that respectively are coupled, directly or indirectly, to one or both of first electrode 102 and second electrode 103.

As explained in greater detail herein, labels 131, 132, 133, and 134 respectively may alter an electrical characteristic of at least one of first polymer chain 111 and second polymer chain in such a manner as to modulate the electrical conductivity or impedance of bridge 110, based upon which modulation the identity of the corresponding nucleotides 121, 122, 123, and 124 may be determined. For example, as explained in greater detail with reference to FIG. 1B, labels 131, 132, 133, and 134 respectively may alter hybridization between first polymer chain 111 and second polymer chain within alteration region 113 in such a manner as to modulate the electrical conductivity or impedance of bridge 110, based upon which modulation the identity of the corresponding nucleotides 121, 122, 123, and 124 may be determined.

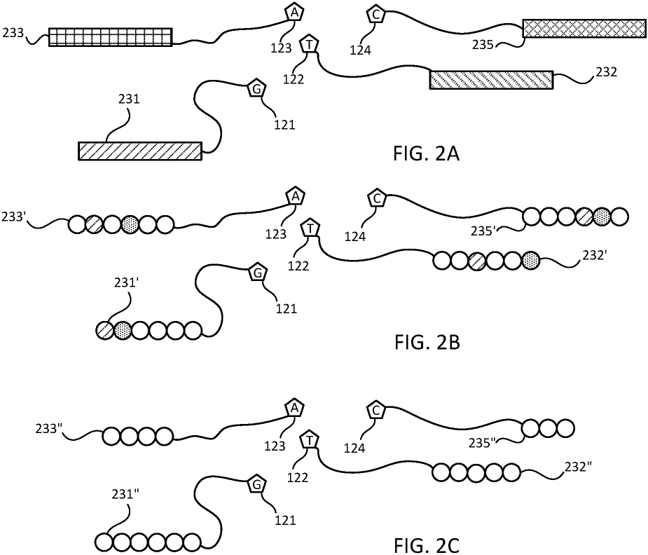

Composition 100 illustrated in FIG. 1A may include any suitable number of nucleotides coupled to corresponding labels, e.g., one or more nucleotides, two or more nucleotides, three or more nucleotides, or four nucleotides. For example, nucleotide 121 (illustratively, G) may be coupled to corresponding label 131, in some examples via linker 135. Nucleotide 122 (illustratively, T) may be coupled to corresponding label 132, in some examples via linker 136. Nucleotide 123 (illustratively, A) may be coupled to corresponding label 133, in some examples via linker 137. Nucleotide 124 (illustratively, C) may be coupled to corresponding label 134, in some examples via linker 138. The couplings between nucleotides and labels, in some examples via linkers which may include the same or different polymer as the labels, may be provided using any suitable methods known in the art, such as n-hydroxysuccinimide (NHS) ester chemistry or click chemistry. Labels 131, 132, 133, and 134 in some examples may include the same type of material as one another, but may differ from one another in at least one respect, e.g., may have different lengths than one another as suggested in FIG. 1A. Alternatively, as described below with reference to FIG. 2A, labels 131, 132, 133, and 134 in some examples may include different materials than one another. As another alternative, labels 131, 132, 133, and 134 in some examples may include the same type of polymer as one another, but may differ from one another in at least one respect, e.g., may have different sequences of monomer units than one another such as in the specific example described with reference to FIG. 2B, or may have different numbers of monomer units than one another such as in the specific example described with reference to FIG. 2C. In some examples, labels 131, 132, 133, and 134 may include the same type of polymer as in alteration region 113, and in some examples may include the same type of polymer as in the remainder of one or both of polymer chains 111, 112. In a manner such as described in greater detail with reference to FIG. 1B, the particular characteristics of labels 131, 132, 133, and 134 may be respectively selected so as to facilitate generation of distinguishable electrical signals, such as currents or voltages, through bridge 110 when those labels respectively alter an electrical characteristic of at least one of first polymer chain 111 and second polymer chain 112. The labels may, but need not necessarily, alter the same electrical characteristic as one another. The labels may, but need not necessarily, alter the electrical characteristic of the same polymer chain as one another. For example, labels may alter different electrical characteristics of different polymer chains, or may alter different electrical characteristics of the same polymer chain, or may alter the same electrical characteristics of different polymer chains, or may alter the same electrical characteristics of the same polymer chain.

Composition 100 illustrated in FIG. 1A includes first polynucleotide 140 and second polynucleotide 150, and polymerase 105 that may add nucleotides of the plurality of nucleotides 121, 122, 123, and 124 to first polynucleotide 140 using at least a sequence of second polynucleotide 150. The labels 131, 132, 133, and 134 corresponding to those nucleotides respectively may alter an electrical characteristic of at least one of the first and second polymer chains 111, 112, e.g., may alter hybridization between first polymer chain 111 and second polymer chain 112, in a manner such as described in greater detail below with reference to FIG. 1B. Detection circuitry 160 may detect a sequence in which polymerase 105 respectively adds the nucleotides 121, 122, 123, and 124 (not necessarily in that order) to first polynucleotide 140 using at least changes in a current through or impedance of bridge 110, the changes being responsive to the alterations in the electrical characteristic using the labels 131, 132, 133, and 134 corresponding to those nucleotides. For example, detection circuitry 160 may apply a voltage across first electrode 102 and second electrode 103, and may detect any current that flows through bridge 110 responsive to such voltage. Or, for example, detection circuitry 560 may flow a constant current through bridge 510, and detect a voltage difference between first electrode 502 and second electrode 503.

At the particular time illustrated in FIG. 1A, none of labels 131, 132, 133, and 134 is in contact with bridge 110, and so a relatively high current may flow through bridge 110. Although nucleotides 121, 122, 123, 124 may diffuse freely through fluid 120 and respective labels 131, 132, 133, 134 may briefly contact bridge 110 as a result of such diffusion, the labels may relatively rapidly dehybridize and so any resulting changes to the electrical conductivity or impedance of bridge 110 are expected to be so short as either to be undetectable, or to be clearly identifiable as not corresponding to addition of a nucleotide to first polynucleotide 140. For example, labels that hybridize as a result of diffusion or due to a polymerase-directed nucleotide incorporation may have identical hybridized lifetimes (statistically speaking). The lifetime is determined by the off rate of the interaction. The off rate is a constant that is governed by the nature of the interaction, temperature, salinity, buffer, and other factors. What distinguishes a true signal from a diffusive one is the percentage of time that the label is bound, and that is determined by the on rate. The on rate increases with the concentration of the label (in contrast to the off rate). For example, concentration corresponds to the probability of finding a molecule in a given volume. The concentration of the label can be orders of magnitude higher for bound nucleotides compared with diffusive ones, because the nucleotide is held in the active site. Thus, the on-rate is much higher. While the labels may dehybridize equally fast in the diffusive and specific states, the specific state results in the labels rebinding very rapidly. After the nucleotide is incorporated, the linker between the label and the nucleotide is severed. As a result, the next time the label dehybridizes, it has the same probability of floating away as the diffusive label.

In comparison, FIG. 1B illustrates a time at which polymerase 105 is adding nucleotide 121 (illustratively, G) to first polynucleotide 140 using at least the sequence of second polynucleotide 150 (e.g., so as to be complementary to a C in that sequence). Because polymerase 105 is acting upon nucleotide 121 to which label 131 is coupled (in some examples via linker 137), such action maintains label 131 at a location that is sufficiently close to bridge 110 for a sufficient amount of time to alter an electrical characteristic of at least one of the first and second polymer chains 111, 112, e.g., to alter hybridization between first polymer chain 111 and second polymer chain 112 within alteration region 113, so as to cause a sufficiently long change in an electrical characteristic, such as electrical conductivity or impedance, of bridge 110 as to be detectable using detection circuitry 160, allowing identification of nucleotide 121 as being added to first polynucleotide 140. Additionally, label 131 may have a property that, when altering an electrical characteristic of at least one of the first and second polymer chains 111, 112, e.g., altering hybridization between first polymer chain 111 and second polymer chain 112, imparts bridge 110 with an electrical characteristic, such as electrical conductivity or impedance, via which detection circuitry 160 may uniquely identify the added nucleotide as 121 (illustratively G) as compared to any of the other nucleotides.

Similarly, label 132 may have a property that, when altering an electrical characteristic of at least one of the first and second polymer chains 111, 112, e.g., altering hybridization between first polymer chain 111 and second polymer chain 112, alters an electrical characteristic, such as electrical conductivity or impedance, of bridge 110 via which detection circuitry 160 may uniquely identify the added nucleotide as 122 (illustratively T) as compared to any of the other nucleotides. Similarly, label 133 may have a property that, when altering an electrical characteristic of at least one of the first and second polymer chains 111, 112, e.g., altering hybridization between first polymer chain 111 and second polymer chain 112, alters an electrical characteristic, such as electrical conductivity or impedance, of bridge 110 via which detection circuitry 160 may uniquely identify the added nucleotide as 123 (illustratively C) as compared to any of the other nucleotides. Similarly, label 134 may have a property that, when altering an electrical characteristic of at least one of the first and second polymer chains 111, 112, e.g., altering hybridization between first polymer chain 111 and second polymer chain 112, alters an electrical characteristic, such as electrical conductivity or impedance, of bridge 110 via which detection circuitry 160 may uniquely identify the added nucleotide as 124 (illustratively C) as compared to any of the other nucleotides.

In the nonlimiting example illustrated in FIGS. 1A-1B, the different lengths of labels 131, 132, 133, and 134 respectively cause alteration region 113 to have different lengths, based upon which the electrical signal between first electrode 102 and second electrode 103 may vary in such a manner that detection circuitry 160 may identify nucleotides 121, 122, 123, 124 respectively coupled to those labels. However, it should be appreciated that labels 131, 132, 133, and 134 may have any suitable respective properties based upon which the electrical signal between first electrode 102 and second electrode 103 may vary in such a manner that detection circuitry 160 may identify nucleotides 121, 122, 123, 124 respectively coupled to those labels.

For example, FIGS. 2A-2C schematically illustrate examples of nucleotides with other labels that alter hybridization within double-stranded polymer bridges. In the nonlimiting example illustrated in FIG. 2A, label 231 includes a first material (suggested by the rectangle having a particular fill) that alters hybridization between first polymer chain 111 and second polymer chain 112. Each of labels 232, 233, and 234 similarly includes a different material (not specifically labeled, but indicated by rectangles having different fills than one another). Such variation in the labels’ materials, when those materials alter hybridization between first polymer chain 111 and second polymer chain 112, provides different and distinguishable signals, e.g., currents or voltages, through bridge 110 based upon which the corresponding nucleotides may be identified.

In the nonlimiting example illustrated in FIG. 2B, label 231′ includes a sequence of two or more signal monomers (suggested by circles having different fills than one another) that respectively hybridize with selected monomers within bridge 110 in such a manner as to alter hybridization between first polymer chain 111 and second polymer chain 112. The signal monomers of label 231′ may be located at any suitable location within the label. Each of labels 232′, 233′, and 234′ similarly includes two or more signal monomers (not specifically labeled, but indicated by circles having different fills than one another), although the particular types and sequences of those monomers vary between labels as intended to be suggested by the different fills of the circles indicating the monomers. Such variation in the labels’ signal monomer types and sequences, when those monomers hybridize with selected monomers within bridge 110, provides different and distinguishable electrical signals, e.g., currents or voltages, through bridge 110 based upon which the corresponding nucleotides may be identified.

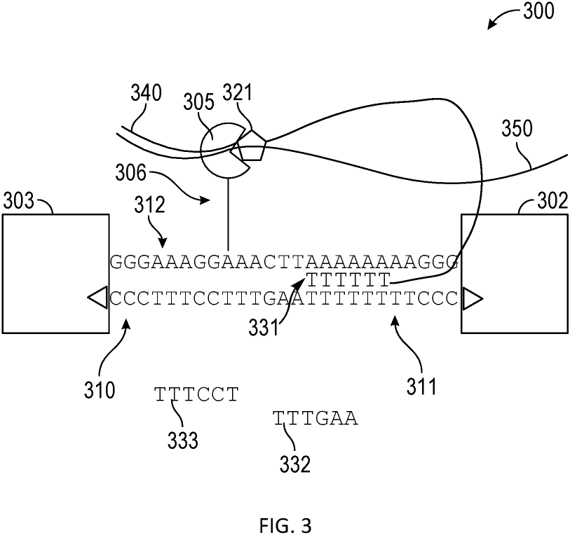

In one nonlimiting example, labels 231′, 232′, 233′, 234′ include respective oligonucleotides having at least partially different sequences than one another. These sequences may hybridize to bridge 110 within alteration region 113 so as to provide a three-stranded “triplex” polynucleotide within alteration region 113. The label’s respective oligonucleotide sequences may hybridize differently than one another with bridge 110 within alteration region 113. For example, signal monomers of label 231′ (suggested by circles having different fills than one another) may be nucleotides that are the same as or different from one another. The signal monomers in the other labels may be nucleotides that are different in sequence or in type, or both, from the first and second signal monomers of the other labels, such that each label 231′, 232′, 233′, 234′ has a unique sequence of first and signal monomers. The respective hybridization between the first and second signal monomers for each label and bridge 110 may provide a particular electrical current or impedance through bridge 110. For example, label 231′ may have a sequence with a particular pair of bases that hybridizes with bases in bridge 110 so as to modulate the electrical conductivity or impedance of bridge 110 to a first level; label 232′ may have a sequence with a particular pair of bases that hybridizes with bases in bridge 110 so as to modulate the electrical conductivity or impedance of bridge 110 to a second level that is different from the first level; label 233′ may have a sequence with a particular pair of bases that hybridizes with bases in bridge 110 so as to modulate the electrical conductivity or impedance of bridge 110 to a third level that is different from the first and second levels; and label 234′ may have a sequence with a particular pair of bases that hybridizes with bases in bridge 110 so as to modulate the electrical conductivity or impedance of bridge 110 to a fourth level that is different from the first, second, and levels. Labels 231′, 232′, 233′, and 243′ in some examples may hybridize with different portions of bridge 110 than one another, in a manner similar to that described with reference to FIG. 3.

Similarly, labels 231′, 232′, 233′, and 234′ respectively may include any suitable combination, number, order, and type of monomer units (e.g., nucleotides) to allow electrical signals from different labels to be detected and distinguished from one another. For example, in FIG. 2C labels 231″, 232″, 233″, and 243″ may have different lengths than one another, e.g., may include any suitable number of monomers that may alter hybridization between first polymer chain 111 and second polymer chain 112, e.g., by hybridizing to bridge 110 within alteration region 113. For example, the labels may include any suitable number of monomers (e.g., nucleotides), e.g., one, two, three, four, five, six, seven, eight, nine, ten, or more than ten monomers. It should be understood that labels 231″, 232″, 233″, and 243″ in some examples also may have different sequences than one another, in a manner such as described with reference to FIG. 2B. Additionally, although the labels illustrated in FIGS. 2A-2C are described as altering hybridization between first and second polymer chains 111, 112, such labels may alter any suitable electrical characteristic or characteristics of one or both of the first polymer chain and the second polymer chain. Additionally, such labels are not limited to use with bridges that include exactly two polymer chains, and indeed may be used with bridges that include a single polymer chain, or more than two polymer chains.

FIG. 3 schematically illustrates an example composition for sequencing that includes a double-stranded polynucleotide bridge and nucleotide labels that alter hybridization between the polynucleotides of the bridge. In the example shown in FIG. 3, composition 300 may be similar to composition 100 described with reference to FIGS. 1A-1B, e.g., includes a substrate (not specifically shown), first electrode 302, second electrode 303, polymerase 305, bridge 310 including first polymer chain 311 and second polymer chain 312, and nucleotide 321 coupled to label 331. Polymerase 305 may be coupled to first polynucleotide chain 311 via linker 306, which may be rigid, and may add nucleotides such as nucleotide 321 to first polynucleotide 340 using at least the sequence of second polynucleotide 350. Composition 300 may include other components such as described with reference to FIGS. 1A-1B, omitted here. It will be appreciated that the particular nucleotide sequences illustrated in FIG. 3 are purely examples, and are not intended to be limiting.

In the example illustrated in FIG. 3, first polynucleotide chain 311 may be coupled to first and second electrodes 302, 303 at points indicated by triangles, and second polynucleotide chain 312 may be hybridized to first polynucleotide chain 311 along the lengths of bridge 310. Label 331 of nucleotide 321 may have a sequence (illustratively, TTTTTT) that hybridizes with bridge 310, providing a first electrical signal through bridge 310. Other labels 332, 333 of other nucleotides (nucleotides not specifically shown) may have different sequences that hybridize with other portions of bridge 310, providing different electrical signals through bridge 310. The different sequences of different labels may be selected so as to provide respective electrical signals through bridge 310 that are distinguishable from one another in a manner such as described with reference to FIGS. 1A-1B.

In some examples the oligonucleotides of labels 231′, 232′, 233′, and 243′ described with reference to FIG. 2B, labels 231″, 232″, 233″, and 243″ described with reference to FIG. 2C, or labels 331, 332, 333 described with reference to FIG. 3 may include modified nucleotides, such as nucleotides with modified backbones (e.g., phosphorothioate DNA), modified sugars (e.g., 2′ o-methyl or 2′ OH (RNA)), modified bases (e.g., methylated bases), or nucleic acid analogs such as peptide-nucleic acids (PNA) or locked nucleic acids (LNA). Such labels, when used with polynucleotide chains 311, 312 (such as DNA, or enantiomeric DNA) may alter hybridization between the polynucleotide chains in such a manner as to detectably change the flow of current or impedance through a bridge including those polynucleotide chains. Modified nucleotides may alter the manner in which polynucleotide chains 311, 312 hybridize with one another. For instance, bulky base modifications in labels may alter the geometry between 311 and 312, thus affecting electrical conduction characteristics. By similar mechanisms, modifications to the sugar or backbone may have similar effects. Any nucleotide based bridges or labels provided herein may include modified nucleotides or nucleic acid analogs such as described with reference to FIGS. 2B, 2C, and 3.

In other examples, labels 131, 132, 133, 134 described with reference to FIGS. 1A-1B or labels 231, 232, 233, 234 described with reference to FIG. 2A may include respective DNA-binding proteins. Such labels, when used with polynucleotide chains 111, 112 (such as DNA, or enantiomeric DNA) may alter hybridization between, or the electrical conduction characteristics of, the polynucleotide chains in such a manner as to detectably change the flow of current or impedance through a bridge including those polynucleotide chains. Nonlimiting examples of DNA-binding proteins that may be used in the present labels include molecular sleds, transcription factors, proteins that function as the binding domain of transcription factors such as designer zinc finger and leucine zippers, catalytically inactive nucleases (e.g,. Hind III, Eco RI), histones, RecA (and other recombinases), and catalytically inactive Crispr-Cas9 and analogs thereof.

In still other examples, labels 131, 132, 133, 134 described with reference to FIGS. 1A-1B or labels 231, 232, 233, 234 described with reference to FIG. 2A may include respective intercalators, such as minor groove binders (MGBs), DNA intercalators, or peptide intercalators. Nonlimiting examples of MGBs include distamycin, netropsin, bisbenzimadazoles, bisamidines, mithramycin, and chromomycin, and their analogs and derivatives. DNA intercalators may include molecules with planar aromatic or heteroaromatic groups capable of stacking between adjacent DNA base pairs. Examples of DNA intercalators that may be used in the present labels include daunomycin, doxorubicin, epirubicin, dactinomycin, ditercalinium, bleomycin, elsamicin A, m-AMSA, mitoxantrone, acridines, and ethidium bromide. For example, ethidium bromide is believed to lengthen the DNA helix, thus altering the electrical conductivity of the DNA helix. Peptide based DNA intercalators may include peptide backbones. An example of a peptide based DNA intercalator is PNA.

In some examples, labels 131, 132, 133, 134 described with reference to FIGS. 1A-1B, or labels 231, 232, 233, 234 described with reference to FIG. 2A, may include respective intertwining alpha helices. Such alpha helix-based labels, when used with double-stranded polymer bridges (e.g., DNA), may alter hybridization between double-stranded chains in such a manner as to detectably change the flow of current or impedance through the bridge. Examples of alpha helices that may be used in the present labels include peptide coiled coils and leucine zippers, such as described in greater detail elsewhere herein.

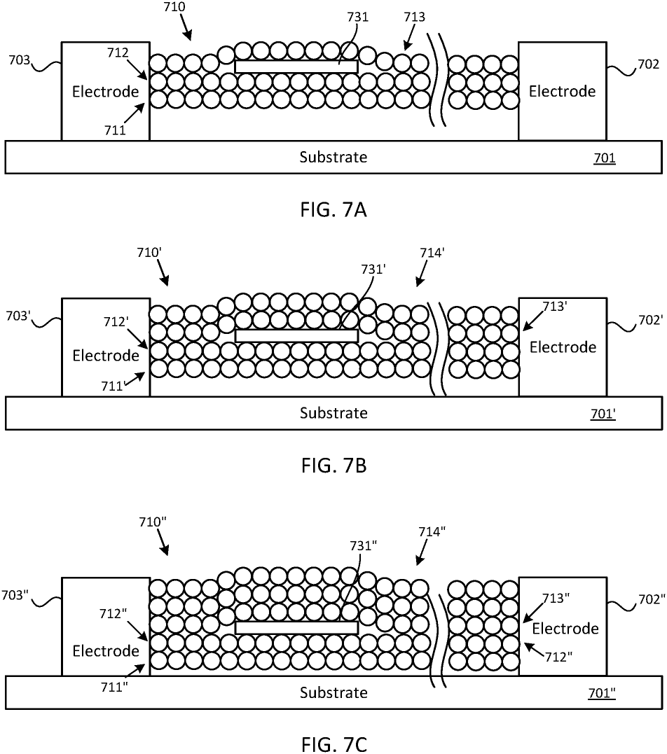

In some examples, bridge 110 described with reference to FIGS. 1A-1B may include any suitable number of polypeptide chains, e.g., two or more polypeptide chains. For example, first and second polymer chains 111, 112 of bridge 110 respectively may include at least first and second polypeptide chains hybridized to one another. Labels 131, 132, 133, 134 described with reference to FIGS. 1A-1B, labels 231, 232, 233, 234 described with reference to FIG. 2A, or label 731 described with reference to FIGS. 7A-7C may include respective proteins, peptides, or intercalators that alter an electrical characteristic of the first and second polypeptides. For example, one or more of the polypeptide chains 111, 112 of bridge 110, and in some examples each of the polypeptide chains of bridge 110, may directly contribute to electron transfer between first electrode 102 and second electrode 103. Without wishing to be bound by any theory, it is believed that such electron transfer may be enabled using, e.g., pi-stacking of aromatic amino acid side chains (such as those of tyrosine, tryptophan, or phenylalanine) in each of the chains. However, other transport mechanisms besides pi-stacking may be used, alone or in combination with pi-stacking. The labels respectively may confer changes in electrical conductivity (an example electrical characteristic) to one or more of the polypeptide chains 111, 112 of bridge 110, for example using formation of a complex such as a dimer, trimer, or higher mer. As such, each of the labels and one or more of the polypeptide chains of bridge 110 may in some examples work together to transfer electrons from first electrode 102 to second electrode 103. The labels may be altered (e.g., via amino acid substitutions of peptide based labels) so as to alter the electrical conductivity of the label-polypeptide chain complex differently than one another, thereby providing different electrical signals via which nucleotides may be identified.

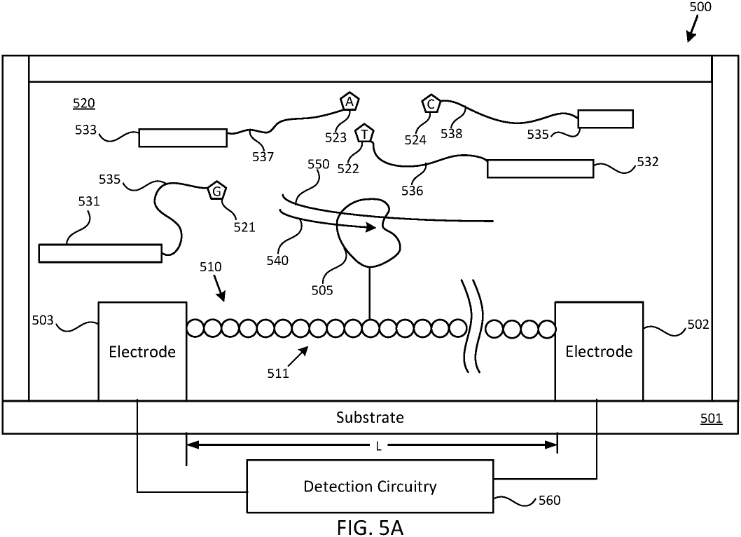

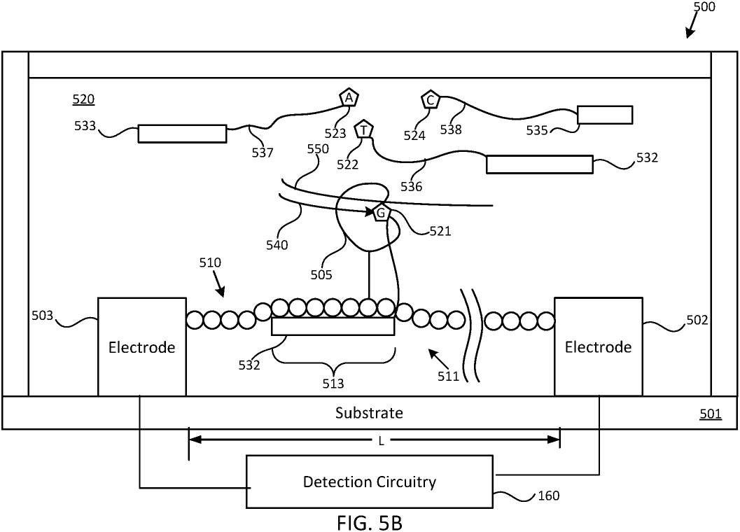

Although examples such as described with reference to FIGS. 1A-1B include double-stranded polymer bridges, it should be appreciated that bridges may be or include single-stranded polymers as well, or may include more than two polymer chains. FIGS. 5A-5B illustrate an example composition 500 for sequencing that includes a single-stranded polymer bridge. Referring now to FIG. 5A, composition 500 includes substrate 501, first electrode 502, second electrode 503, polymerase 504, bridge 510, nucleotides 521, 522, 523, and 524, labels 531, 532, 533, and 534 respectively coupled to those nucleotides, first polynucleotide 540, second polynucleotide 550, and detection circuitry 560. Polymerase 505 is in proximity of bridge 510, substrate 501 may support first electrode 502 and second electrode 503, and components of composition 500 may be enclosed within a flow cell (e.g., having walls 561, 562, 562) filled with fluid 520 in which nucleotides 521, 522, 523, and 524 (with associated labels), polynucleotides 540, 550, and suitable reagents may be carried, in a manner such as described with reference to FIGS. 1A-1B.

Bridge 510 may span the space between first electrode 502 and second electrode 503, and may include polymer chain 511 (the circles within the polymer chain being intended to suggest monomer units that are coupled to one another along the length of the polymer chain). Polymer chain 511 may have length that is approximately the same as length L of the space between first electrode 502 and second electrode 503 or otherwise permits polymer chain 511 to span the space between first electrode 502 and second electrode 503, e.g., such that polymer chain 511 may be coupled directly to each of first electrode 502 and second electrode 503 (e.g., via respective bonds). In some examples, polymer chain 511 may include a polypeptide chain. The polypeptide chain may be helical in some examples. For example, helical polypeptides are believed to be good electron mediators that may transfer electrons over relatively long distances. Without wishing to be bound by any theory, it is believed that polypeptides may conduct using an electron tunneling mechanism, a hopping mechanism, or both. In an electron tunneling mechanism, electrons may travel through the molecular orbitals of the polypeptide chain, e.g., through aromatic amino acids such as tyrosine, tryptophan, or phenylalanine. In a hopping mechanism, charged particles (positive or negative) may hop through the polypeptide chain. The polypeptide chain may form a variety of structures in addition to those discussed, including a beta strand. The polypeptide chain may include any suitable combination of natural amino acids and non-natural amino acids. Large aromatic residues (tyrosine, phenylalanine, tryptophan) and 0-branched amino acids (threonine, valine, isoleucine) are favored to be found in 0-strands in the middle of 0-sheets, and the aromatic residues in particular would be expected to contribute to conductivity via the mechanisms discussed above.

In another specific, nonlimiting example, the polypeptide chain includes the sequence GFPRFAGFP (SEQ ID NO: 1), which is believed to have a left-handed helical backbone conformation that allows extended F aromatic group stacking to provide pi-pi conjugation via which electrons may flow. Other combinations and ordering of aromatic residues could be used to create peptides capable of different magnitudes of conductivity, particularly when complexed with a second peptide to form a coiled coil. In some examples, a label for use with such a chain may include a second identical, or approximately identical, copy of the same sequence, or a similar polypeptide that lacks an F or replaces F with another aromatic residue, such as Y, that electrically conducts differently than F. In such a manner, combining two of the same to form a coiled coil, or two different helices to form a coiled coil, may result in different conductivity relative to the monomer wire, thus permitting identification of the second label, and ultimately the identity of the nucleotide linked to the label. The foregoing principal extends to more than two labels such that 4 nucleotides can be encoded.

In yet another specific, nonlimiting example, one or more of the polypeptide chains includes the sequence FKEFAKL FKEFAKL FHKFAKL (SEQ ID NO: 2), which is believed to self-assemble into fibrils including multiple copies of such sequence, such fibrils similarly being electrically conductive. One instance of such sequence may be provided in bridge 510, and another instance of such sequence may be provided in a first label for use with such bridge and may be expected to self-assemble with the sequence in the bridge in such a manner as to alter the electrical conductivity of the bridge, e.g., by increasing electrical conductivity of the bridge. A second label for use with such bridge may include the sequence LKELAKL LKELAKL LHELAKL (SEQ ID NO: 3), which is believed to self-assemble into fibrils including multiple copies of such sequence, such fibrils being electrically nonconductive. This sequence in the second label may be expected to self-assemble with the sequence FKEFAKL FKEFAKL FHKFAKL (SEQ ID NO: 2) in the bridge in such a manner as to alter the electrical conductivity of the bridge, e.g., by providing an electrical conductivity of the bridge that is less than that provided by the first label sequence FKEFAKL FKEFAKL FHKFAKL (SEQ ID NO: 2). Other combinations and ordering of aromatic residues in both the wire and label could be used to create peptides capable of different magnitudes of conductivity, particularly when complexed to form coiled coils. In such a manner, combining two of the same, or two different helices to form coiled coils, may result in different conductivity relative to the monomer wire, thus permitting identification of the second label, and ultimately the identity of the nucleotide linked to the label. The foregoing principal extends to more than two labels such that 4 nucleotides can be encoded.

In still another specific, nonlimiting example, bridge 510 may include a PilA protein, such as may occur in natural microbial pili, which are believed to be electrically conductive. PilA proteins of G. sulfurreducens are believed to include a coiled-coil motif that forms an electrically conductive nanowire.

As explained in greater detail below with reference to FIG. 5B, labels 531, 532, 533, and 534 respectively may alter an electrical characteristic of polymer chain 511 within alteration region 513 in such a manner as to modulate the electrical conductivity or impedance of bridge 510, based upon which modulation the identity of the corresponding nucleotides 521, 522, 523, and 524 may be determined. Composition 500 illustrated in FIG. 5A may include any suitable number of nucleotides coupled to corresponding labels, e.g., one or more nucleotides, two or more nucleotides, three or more nucleotides, or four nucleotides, in a manner similar to that described with reference to FIGS. 1A-1B. For example, nucleotide 521 (illustratively, G) may be coupled to corresponding label 531, in some examples via linker 535. Nucleotide 522 (illustratively, T) may be coupled to corresponding label 532, in some examples via linker 536. Nucleotide 523 (illustratively, A) may be coupled to corresponding label 533, in some examples via linker 537. Nucleotide 524 (illustratively, C) may be coupled to corresponding label 534, in some examples via linker 538. In a manner such as described in greater detail with reference to FIG. 5B, the particular characteristics of labels 531, 532, 533, and 534 may be respectively selected so as to facilitate generation of distinguishable electrical signals, such as currents or voltages, through bridge 510 when those labels alter the electrical characteristic within polymer chain 511.

Composition 500 illustrated in FIG. 5A includes first polynucleotide 540 and second polynucleotide 550, and polymerase 505 that may add nucleotides of the plurality of nucleotides 521, 522, 523, and 524 to first polynucleotide 540 using at least a sequence of second polynucleotide 550. The labels 531, 532, 533, and 534 corresponding to those nucleotides respectively may alter an electrical characteristic of polymer chain 511 in a manner such as described in greater detail below with reference to FIG. 5B. Detection circuitry 560 may detect a sequence in which polymerase 505 respectively adds the nucleotides 521, 522, 523, and 524 (not necessarily in that order) to first polynucleotide 540 using at least changes in a current through or impedance of bridge 510, the changes being responsive to the alterations in an electrical characteristic of the polymer chain using the labels 531, 532, 533, and 534 corresponding to those nucleotides. For example, detection circuitry 560 may apply a voltage across first electrode 502 and second electrode 503, and may detect any current that flows through bridge 510 responsive to such voltage. Or, for example, detection circuitry 560 may flow a constant current through bridge 510, and detect a voltage difference between first electrode 502 and second electrode 503.

At the particular time illustrated in FIG. 5A, none of labels 531, 532, 533, and 534 are in contact with bridge 510, and so a relatively high (or low) current may flow through bridge 510. In comparison, FIG. 5B illustrates a time at which polymerase 505 is adding nucleotide 521 (illustratively, G) to first polynucleotide 540 using at least the sequence of second polynucleotide 550 (e.g., so as to be complementary to a C in that sequence). Because polymerase 505 is acting upon nucleotide 521 to which label 531 is coupled (in some examples via linker 537), such action maintains label 531 at a location that is sufficiently close to bridge 510 for a sufficient amount of time to cause a sufficiently long change in an electrical characteristic, such as electrical conductivity or impedance, of bridge 510 as to be detectable using detection circuitry 560, allowing identification of nucleotide 521 as being added to first polynucleotide 540. For example, label 531 may cause deformation of polymer chain 511 in a manner such as illustrated in FIG. 5B (e.g., may induce twisting, kinking, elongation, or other conformation change), or may alter an electrically conductive state of polymer chain 511, such that a lower (or higher) current flows through bridge 510. In one specific example, polymer chain 511 includes a first helical polypeptide and label 531 includes a second helical polypeptide that forms a dimer with the first helical polypeptide that changes an electrical characteristic of bridge 510. For example, the second helical polypeptide may change the conformation of the first polypeptide in such a manner as to change an electrical conductivity of the first polypeptide or to alter an electrical environment of the amino acids in the first polypeptide. Illustratively, electrical conductivity of a polypeptide chain may be expected to be tunable using the location(s) of tryptophan residue(s). For example, electrical conductivity of a polypeptide chain may be expected to be higher when a tryptophan residue is located near either end of the polypeptide chain, and may expected to be lower when the tryptophan residue is located near the middle of the polypeptide chain. In such a manner, the respective electrical conductivities of first and second helical polypeptides may be tuned so as to provide distinguishable electrical signals, and function as different labels when associated with a bridge, thus permitting identification of the nucleotides coupled to such labels. Alternatively, such tunable peptides may be tuned for use in bridges, and their electrical conductivity modulated using labels.

Labels 531, 532, and 533 similarly may have respective properties that, when altering an electrical characteristic of first polymer chain 511, changes electrical conductivity or impedance of bridge 510, via which detection circuitry 560 may uniquely identify the added nucleotide as compared to any of the other nucleotides. In the nonlimiting example illustrated in FIGS. 5A-5B, the different lengths of labels 531, 532, 533, and 534 respectively cause alteration region 513 to have different lengths, based upon which the electrical signal between first electrode 502 and second electrode 503 may vary in such a manner that detection circuitry 560 may identify nucleotides 521, 522, 523, 524 respectively coupled to those labels. However, it should be appreciated that labels 531, 532, 533, and 534 may have any suitable respective properties based upon which the electrical signal between first electrode 502 and second electrode 503 may vary in such a manner that detection circuitry 560 may identify nucleotides 521, 522, 523, 524 respectively coupled to those labels.

In some examples, one or more of the labels described with reference to FIGS. 1A-1B, FIG. 2A, FIGS. 5A-5B, or FIGS. 7A-7C may include respective intercalators, such as peptides that intercalate into DNA. Such peptide-based labels, which in some examples may be used with polynucleotide chains or with polypeptide chains, may alter hybridization between the chains in such a manner as to detectably change the flow of current or impedance through a bridge including those chains, or otherwise may alter an electrical characteristic of one or more of the chains in such a manner as to detectably change the flow of current through a bridge including that chain. One nonlimiting example of a peptide that may be included in a label for use with ssDNA or multiple-stranded DNA (e.g., dsDNA or triple-stranded DNA) is the heptapeptide KGKGKGK (SEQ ID NO: 4), which binds to the DNA sequence poly(dG-d5meC) and may convert that DNA sequence from the B conformation to the Z conformation. Such a conformational change may be expected to be concomitant with a conductivity change in a DNA bridge. Additionally, as noted above, PNA may be included in the present labels. The PNA may, for example, form a triplex with a dsDNA bridge. Other example labels that may form triplexes with a dsDNA bridge include LNA, 2′-O-methyl ribonucleotides, and RNA. Example labels that may form triplexes with a double-stranded RNA bridge include single-stranded RNA.

In some examples, one or more of one or more of the labels described with reference to FIGS. 1A-1B, FIG. 2A, FIGS. 5A-5B, or FIGS. 7A-7C may include respective intertwining alpha helices. Such alpha helix-based labels, when used with multiple-stranded (e.g., double-stranded) polymer bridges (e.g., polynucleotide or polypeptide), may alter hybridization between the polymer chains in such a manner as to detectably change the impedance or flow of current through the bridge. Or, when such labels are used with bridges that include a single-stranded polymer chain (e.g., single polypeptide chains), may alter an electrical characteristic of that chain in such a manner as to detectably change the flow of current through a bridge including that chain. Examples of alpha helices that may be used in the present labels include peptide coiled coils and leucine zippers. For example, a set of peptides of differing lengths and compositions may be suitably designed so as to interact with one another in various combinations to form coiled coil heterodimer regions of, e.g., about 21 residues, about 24 residues, or about 28 residues of varying stabilities. The resulting coiled coils may have dissociation constants in the micromolar to sub-nanomolar range, thus displaying a broad range of tunable stabilities.

In some examples, one or more of the labels described with reference to FIGS. 1A-1B, FIG. 2A, FIGS. 5A-5B, or FIGS. 7A-7C may include respective peptides or proteins. Such peptide-based or protein-based labels, when used with polypeptide chain(s), may alter an electrical characteristic of the chain in such a manner as to detectably change the flow of current or impedance through a bridge including that chain. For example, one or more of the labels may include a sequence of peptides that, together with a polypeptide chain, form a leucine zipper, where one alpha helix chain of the zipper is provided by the polypeptide chain and the other half of the zipper is provided by the label. Example peptides that may be expected to interact with dsDNA or tsDNA (triple stranded DNA) include, but are not limited to, alpha-helical peptides such as Ac-(LRAL)3-OH (SEQ ID NO: 5), B-turn peptides such as gramicidin, antiparallel B-sheet peptides such as Ac-(KL)7-OH (SEQ ID NO: 6), and beta-hairpin peptides such as Ac-(LR)5LFPV(RL)5-OH (SEQ ID NO: 7). For example, Ac-(LRAL)3-OH (SEQ ID NO: 5) and Ac-(LR)5LFPV(RL)5-OH (SEQ ID NO: 7) are expected to interact with the dsDNA fragment:

5′-GCTAAAAAGAGAGAGAGATCG-3′ (SEQ ID NO: 8)

3′-CGATTTTTCTCTCTCTCTAGC-5′ (SEQ ID NO: 9)

while Gramicidin and Ac-(LRAL)3-OH (SEQ ID NO: 5) are expected to bind to and stabilize tsDNA. Such protein-DNA interactions may be expected to alter the conductivity of a DNA-based bridge, for example, by changing the shape of the DNA.

In another example, one or more of the labels described with reference to FIGS. 1A-1B, FIG. 2A, FIGS. 5A-5B, or FIGS. 7A-7C may include a sequence of peptides that, together with a polypeptide chain, form a coiled coil, where one coil is provided by the polypeptide chain and the other coil is provided by the label. For example, coiled coils may be formed when two or more α-helices self-assemble by winding around each other to form a left-handed supercoil. Dimers, trimers, and tetramers readily can be designed, and even larger coiled-coils of up to seven helices readily can be designed. Coiled coils may include a specific packing architecture known as “knobs-into-holes” (KIH) whereby the side-chains of hydrophobic residues act as “knobs” and pack into “holes” formed by four residues from a neighboring helix. Some coiled coils also contain a heptad repeat sequence pattern that can repeat. A heptad may include seven amino acids which may be labeled as, “abcdefg,” where hydrophobic residues reside at the “a” and “d” positions, resulting in a hydrophobic/hydrophilic pattern of the form, “(HPPHPPP)n.” Localizing hydrophobic amino acids three and four residues apart may result in such hydrophobic amino acids residing on the same face of the helix, so burial of this hydrophobic face may be a driving force for coiled-coil formation.

Other examples of elements that may be used as labels for use with a polypeptide chain include dsDNA or ssDNA (which are negatively charged and thus may bind to a positively charged peptide wire); or anti-peptide nucleic acid aptamers. For example, anti-peptide nucleic acid aptamers may be readily selected against small peptide targets, ranging from about 5 residues to about 20 residues. Such a peptide may be provided, for example, as an electrically conductive bridge, while the anti-peptide nucleic acid aptamer may be provided as a label that may be expected to change the electrical conductance of such a bridge. In some examples, aptamers may be selected, from a random pool of nucleic acid sequences, that recognize the peptide epitope. As one example, one such epitope may include a peptide corresponding to residues 34-50 of the Rev protein of HIV-1. The selected aptamers may bind stably and specifically to the peptide epitope, for instance with Kd values of 19-36 nM.

As noted further above, in still other examples, the present bridges may include more than two polymer chains, e.g., may include three, four, five, or more than five polymer chains. FIGS. 7A-7C illustrate example polymer bridges including more than two polymer chains. FIG. 7A illustrates polymer bridge 710 extending between first and second electrodes 702, 703 and including first polymer chain 711, second polymer chain 712, and third polymer chain 713. FIG. 7B illustrates polymer bridge 710′ extending between first and second electrodes 702′, 703′ and including first polymer chain 711′, second polymer chain 712′, third polymer chain 713′, and fourth polymer chain 714′. FIG. 7C illustrates polymer bridge 710″ extending between first and second electrodes 702″, 703″ and including first polymer chain 711″, second polymer chain 712″, third polymer chain 713″, and fourth polymer chain 714″. In some examples, one or more, two or more, or all of the polymer chains illustrated in each of FIGS. 7A-7C may include a polypeptide. In some examples, one or more, two or more, or all of the polymer chains, e.g., one or more, two or more, or all of the polypeptides, may be helical, or may form a beta-strand. For example, in a manner such as described above with reference to FIG. 5A, helical polypeptides are believed to be good electron mediators and may transfer electrons over relatively long distances. Each of the polymer (e.g., polypeptide) chains illustrated in FIGS. 7A-7C may include any suitable combination of natural amino acids and non-natural amino acids.

In one specific, nonlimiting example, helical peptides having an alternating amino acid sequence of Ala-Aib (alanine-2-aminoisobutyric acid) sequence, ranging in lengths from 8mer to 16mer to 24mer, may be used as a polypeptide chain in one of the present bridges. Without wishing to be bound by any theory, a hopping mechanism may be responsible for long-range electron transfer in such polypeptide chains.

In another specific, nonlimiting example, one or more of the polypeptide chains includes the sequence GFPRFAGFP (SEQ ID NO: 1), which is believed to have a left-handed helical backbone conformation that allows extended F aromatic group stacking to provide pi-pi conjugation via which electrons may flow. In some examples, a label for use with such a chain may include a second identical, or approximately identical, copy of the same sequence, or a similar polypeptide that lacks an F or replaces F with another aromatic residue, such as Y, that electrically conducts differently than F.

In yet another specific, nonlimiting example, one or more of the polypeptide chains includes the sequence FKEFAKL FKEFAKL FHKFAKL (SEQ ID NO: 2), which is believed to self-assemble into fibrils including multiple copies of such sequence, such fibrils similarly being electrically conductive. One or more instances of such sequence may be provided in bridge 710, 710′, 710″, and another instance of such sequence may be provided in a first label for use with such bridge and may be expected to self-assemble with the sequence in the bridge in such a manner as to alter the electrical conductivity of the bridge, e.g., by increasing electrical conductivity of the bridge. A second label for use with such bridge may include the sequence LKELAKL LKELAKL LHELAKL (SEQ ID NO: 3), which is believed to self-assemble into fibrils including multiple copies of such sequence, such fibrils being electrically nonconductive. This sequence in the second label may be expected to self-assemble with one or more of the sequence(s) FKEFAKL FKEFAKL FHKFAKL (SEQ ID NO: 2) in the bridge in such a manner as to alter the electrical conductivity of the bridge, e.g., by providing an electrical conductivity of the bridge that is less than that provided using the first label sequence FKEFAKL FKEFAKL FHKFAKL (SEQ ID NO: 2).

In another specific, nonlimiting example, one or more of the polypeptide chains includes the sequence ELKAIAQEFKAIAKEFKAIAFEFKAIAQK (SEQ ID NO: 10), which is believed to self-assemble into electrically conductive hexamer coils in which the spacing and arrangement of aromatic side chains is believed to preclude pi-stacking as a mechanism for electron transport.

Labels such as described with reference to FIGS. 1A-1B or 2A-2C respectively may alter an electrical characteristic of bridge 710, 710′, or 710″. For example, label 731 illustrated in FIG. 7A may confer an altered electrical conductivity to at least one of the polypeptide chains of bridge 710 by forming a dimer with one of the polypeptide chains, by forming a trimer with two of the polypeptide chains, or by forming a quatromer with all three of the polypeptide chains. Or, for example, label 731′ illustrated in FIG. 7B may confer an altered electrical conductivity to at least one of the polypeptide chains of bridge 710′ by forming a dimer with one of the polypeptide chains, by forming a trimer with two of the polypeptide chains, by forming a quatromer with three of the polypeptide chains, or by forming a pentamer with all four of the polypeptide chains. Or, for example, label 731″ illustrated in FIG. 7C may confer an altered electrical conductivity to at least one of the polypeptide chains of bridge 710″ by forming a dimer with one of the polypeptide chains, by forming a trimer with two of the polypeptide chains, by forming a quatromer with three of the polypeptide chains, by forming a pentamer with all four of the polypeptide chains, or by forming a hexamer with all five of the polypeptide chains.

Labels 731, 731′, 731″ may include any suitable element that detectably alters an electrical characteristic of bridges 710, 710′, 710″, respectively. In some examples, labels 731, 731′, 731″ are peptide intercalators. One example of a peptide intercalator that may be used in a label is a coil, which may be used for example with a polypeptide based bridge that itself includes two or more polypeptide chains that form a coiled coil. The coil of the label may form a bundle (e.g., a triplex) with the coiled coil of the polypeptide bridge, and thus may detectably alter an electrical characteristic of the bridge.

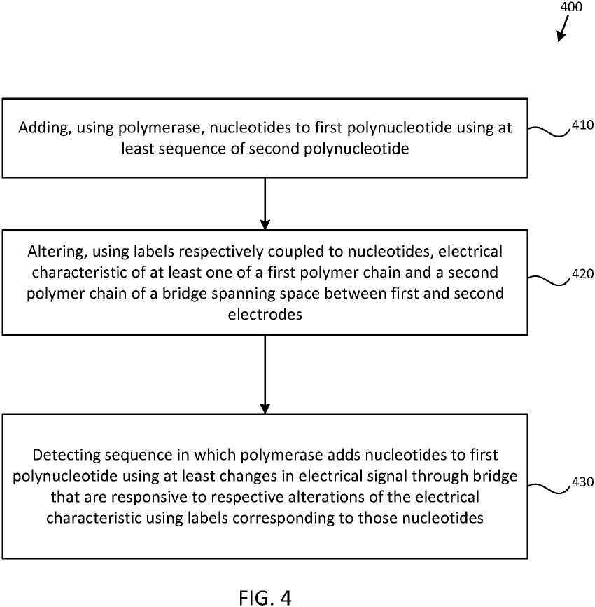

Compositions such as described with reference to FIGS. 1A-1B, FIGS. 2A-2C, FIG. 3, and FIGS. 7A-7C may be used in any suitable method for sequencing. FIG. 4 illustrates an example flow of operations in a method 400 for sequencing using a double-stranded polymer bridge and nucleotide labels that alter an electrical characteristic of at least one the polymer strand of the bridge. Method 400 includes adding, using a polymerase, nucleotides to a first polynucleotide using at least a sequence of a second polynucleotide (operation 410). For example, polymerase 105 described with reference to FIGS. 1A-1B may add each of nucleotides 121, 122, 123, and 124 to first polynucleotide 140 using at least the sequence of second polynucleotide 150. Or, for example, polymerase 305 described with reference to FIG. 3 may add nucleotide 321 and other nucleotides to first polynucleotide 340 using at least the sequence of second polynucleotide 350 (other nucleotides not specifically shown). The compositions illustrated in FIGS. 7A-7C may include a polymerase (not specifically) which similarly may add nucleotides to a first polynucleotide using at least a sequence of a second polynucleotide.

Method 400 illustrated in FIG. 4 may include altering, using labels respectively coupled to the nucleotides, an electrical characteristic of at least one of first and second polymer chains of a bridge spanning a space between first and second electrodes (operation 420). For example, any of labels 131, 132, 133, 134 described with reference to FIGS. 1A-1B, labels 231, 232, 233, 234 described with reference to FIG. 2A, labels 231′, 232′, 233′, 234′ described with reference to FIG. 2B, labels 231″, 232″, 233″, 234″ described with reference to FIG. 2C respectively may be coupled to nucleotides 121, 122, 123, and 124. As polymerase 105 respectively adds those nucleotides to first polynucleotide 140, the labels coupled to those nucleotides respectively may alter an electrical characteristic of at least one of first polymer chain 111 and second polymer chain 112 within bridge 110 which spans the space between first electrode 102 and second electrode 103. Or, for example, label 331 described with reference to FIG. 3 may be coupled to nucleotide 321, and other labels such as labels 332, 333 may be coupled to other nucleotides (other nucleotides not specifically shown). As polymerase 305 respectively adds those nucleotides to first polynucleotide 340, the labels coupled to those nucleotides respectively may hybridize to bridge 310 so as to alter hybridization between first polynucleotide chain 311 and second polynucleotide chain 312 of bridge 310 which spans the space between first electrode 302 and second electrode 303. For the compositions illustrated in FIGS. 7A-7C, labels coupled to nucleotides similarly may alter an electrical characteristic of one or more of the polymer chains of those compositions.

Referring again to FIG. 4, method 400 may include detecting a sequence in which the polymerase adds the nucleotides to the first polynucleotide using at least changes in electrical signal, e.g., current or voltage, through the bridge that are responsive to respective alterations of the electrical characteristic using the labels corresponding to those nucleotides (operation 440). For example, detection circuitry 160 described with reference to FIGS. 1A-1B may detect changes in electrical signal through bridge 110 responsive to respective alterations using labels 131, 132, 133, and 134, or using labels 231, 232, 233, 234 described with reference to FIG. 2A, or using labels 231′, 232′, 233′, 234′ described with reference to FIG. 2B, or using labels 231″, 232″, 233″, 234″ described with reference to FIG. 2C. Similar detection circuitry (not specifically illustrated) may detect changes in electrical signal through bridge 310, illustrated in FIG. 3, responsive to respective hybridizations between labels 331, 332, 333 (and other similar labels) and bridge 310. Similar detection circuitry (not specifically illustrated) may detect changes in electrical signal through bridges 710, 710′, 710″, respectively illustrated in FIGS. 7A-7C, responsive to respective hybridizations between labels 731, 731′, 731″ (and other similar labels) and the respective bridges.

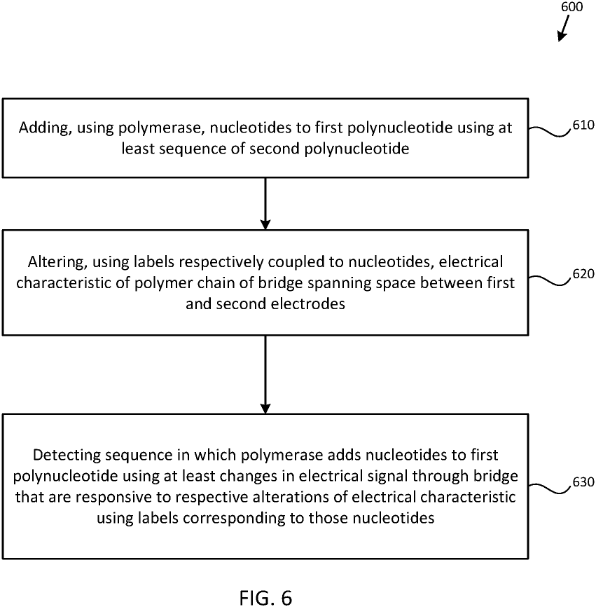

Additionally, compositions such as described with reference to FIGS. 5A-5B may be used in any suitable method for sequencing. FIG. 6 illustrates an example flow of operations in a method 600 for sequencing using a single-stranded polymer bridge and nucleotide labels that alter an electrical characteristic of the polymer strand of the bridge. Method 600 includes adding, using a polymerase, nucleotides to a first polynucleotide using at least a sequence of a second polynucleotide (operation 610). For example, polymerase 505 described with reference to FIGS. 5A-5B may add each of nucleotides 521, 522, 523, and 526 to first polynucleotide 560 using at least the sequence of second polynucleotide 550.

Method 600 illustrated in FIG. 6 may include altering, using labels respectively coupled to the nucleotides, an electrical characteristic of a polymer chain of a bridge spanning a space between first and second electrodes (operation 620). For example, any of labels 531, 532, 533, 534 described with reference to FIGS. 5A-5B respectively may be coupled to nucleotides 521, 522, 523, and 524. As polymerase 505 respectively adds those nucleotides to first polynucleotide 540, the labels coupled to those nucleotides respectively may alter an electrical characteristic of polymer chain 511 within bridge 510 which spans the space between first electrode 502 and second electrode 503.

Referring again to FIG. 6, method 600 may include detecting a sequence in which the polymerase adds the nucleotides to the first polynucleotide using at least changes in electrical signal, e.g., current or voltage, through the bridge that are responsive to respective alterations of the electrical characteristic using the labels corresponding to those nucleotides (operation 640). For example, detection circuitry 560 described with reference to FIGS. 5A-5B may detect changes in electrical signal through bridge 510 responsive to respective alterations using labels 531, 532, 533, and 534.

Any suitable modifications may be made to any of the compositions and methods provided herein. In some examples, compositions 100, 300, or 500 may be modified such that any suitable polymers therein (such as polynucleotides of the first and second polymer chains or oligonucleotides of the labels, or both) include non-naturally occurring polynucleotides, such as non-naturally occurring DNA, e.g., enantiomeric DNA. Such non-naturally occurring polynucleotides may not hybridize with any naturally occurring polynucleotides in the compositions, for example, the first and second polynucleotides being acted upon using the polymerase, thus minimizing, and in some instances even inhibiting, any interference that otherwise may result from such hybridization.

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

What is claimed is:

1. A composition, comprising:
first and second electrodes separated from one another by a space;
a bridge spanning the space between the first and second electrodes,
the bridge comprising first and second polymer chains hybridized to one another;
first and second polynucleotides;
a plurality of nucleotides, each nucleotide coupled to a corresponding label;
a polymerase to add nucleotides of the plurality of nucleotides to the first polynucleotide using at least a sequence of the second polynucleotide,
the labels corresponding to those nucleotides respectively altering an electrical characteristic of at least one of the first and second polymer chains; and
detection circuitry to detect a sequence in which the polymerase adds the nucleotides to the first polynucleotide using at least changes in an electrical signal through the bridge, the changes being responsive to alteration of the electrical characteristic using the labels corresponding to those nucleotides.

2. The composition of claim 1, wherein the first and second polymer chains respectively comprise first and second polynucleotides hybridized to one another.

3. The composition of claim 2, wherein the labels comprise respective oligonucleotides that alter the hybridization between the first and second polynucleotides.

4. The composition of claim 3, wherein the oligonucleotides alter the hybridization in different locations than one another.

5. The composition of claim, wherein the oligonucleotides alter the hybridization in regions of different length.

6. The composition of claim 2, wherein the polynucleotides of the first and second polymer chains and the oligonucleotides of the labels comprise non-naturally occurring DNA.

7. The composition of claim 6, wherein the non-naturally occurring DNA comprises enantiomeric DNA.

8. The composition of claim 3, wherein the oligonucleotides comprise modified nucleotides.

9. The composition of claim 8, wherein the modified nucleotides have modified backbones, modified sugars, or modified bases.

10. The composition of claim 3, wherein the oligonucleotides comprise nucleic acid analogs selected from the group consisting of PNA and LNA.

11. The composition of claim 2, wherein the first and second polynucleotides comprise DNA, and wherein the labels comprise proteins that interact with the DNA.

12. The composition of claim 1, wherein the labels comprise DNA intercalators.

13. The composition of claim 1, wherein the labels comprise minor groove binders.

14. The composition of claim 1, wherein the labels comprise peptide intercalators.

15. The composition of claim 1, wherein the labels comprise intertwining alpha helices.

16. The composition of claim 1, wherein the first and second polymer chains respectively comprise first and second polypeptides hybridized to one another.

17. The composition of claim 16, wherein each of the labels comprises a protein, peptide, or intercalator that alters an electrical characteristic of at least one of the first and second polypeptides.

18. A method, the method comprising:
adding, using a polymerase, nucleotides to a first polynucleotide using at least a sequence of a second polynucleotide;
altering, using labels respectively coupled to the nucleotides, an electrical characteristic of at least one of a first polymer chain and a second polymer chain of a bridge spanning a space between first and second electrodes; and
detecting a sequence in which the polymerase adds the nucleotides to the first polynucleotide using at least changes in electrical signal through the bridge that are responsive to respective alterations of the electrical characteristic using the labels corresponding to those nucleotides.

19-34. (canceled)

35. A composition, comprising:
first and second electrodes separated from one another by a space;
a bridge spanning the space between the first and second electrodes, the bridge comprising a polymer chain;
first and second polynucleotides;
a plurality of nucleotides, each nucleotide coupled to a corresponding label;
a polymerase to add nucleotides of the plurality of nucleotides to the first polynucleotide using at least a sequence of the second polynucleotide,
the labels corresponding to those nucleotides respectively altering an electrical characteristic of the polymer chain; and
detection circuitry to detect a sequence in which the polymerase adds the nucleotides to the first polynucleotide using at least changes in an electrical signal through the bridge, the changes being responsive to alteration of the electrical characteristic using the labels corresponding to those nucleotides.

36-38. (canceled)

39. A method, the method comprising:
adding, using a polymerase, nucleotides to a first polynucleotide using at least a sequence of a second polynucleotide;
altering, using labels respectively coupled to the nucleotides, an electrical characteristic of a polymer chain of a bridge spanning a space between first and second electrodes; and
detecting a sequence in which the polymerase adds the nucleotides to the first polynucleotide using at least changes in electrical signal through the bridge that are responsive to respective alterations of the electrical characteristic using the labels corresponding to those nucleotides.

40-42. (canceled)
